(19) United States
(12) Patent Application Publication
(10) Pub. No.: US 2008/0202516 A1
(43) Pub. Date: Aug. 28, 2008

(54) Portable pulmonary body core cooling and heating system

(76) Inventor: Mark R. Harvie, Milton, VT (US)

Correspondence Address: Law Offices Eric R. Benson, Esq., 6A Hillside Lane, Westford, VT, 05494-9769, US

(21) Appl. No.: 12/070,435

(22) Filed: Feb. 19, 2008

Related U.S. Application Data
(60) Provisional application No. 60/903,202, filed on Feb. 23, 2007.

(51) Int. Cl. A61M 16/00; A61M 16/06

(52) U.S. Cl. 128/204.15; 128/204.17; 128/206.21

(57) ABSTRACT

This invention relates to a fully adjustable Portable Pulmonary Body Core Cooling and Heating System specifically designed to provide several hours of high efficiency cooling or heating when worn and operated by a user. This combination Portable Pulmonary Body Core Cooling and Heating System invention is capable of delivering several hours of high efficiency personal cooling or heating without the use of caustic or toxic chemicals with virtually no risk of injury associated with its use. This Portable Pulmonary Body Core Cooling and Heating System invention utilizes the user's own pulmonary system to regulate core body temperature by introducing controlled temperature air into the user's lungs by their own breathing process.

### BACKGROUND ART

The human thermo-regulatory system maintains the human body core temperature within 0.2° C. of 37° C. When this core temperature deviates significantly metabolic deterioration takes place and even death may result. Various studies of surgeries have shown that a 1° C. to 2° C. hypothermia is empirically associated with increased mortality rates with triple the rate of cardiac episodes, triple the incidence of surgically related infections, and prolonged hospitalization. Hypothermia is also implicated in significant increases of surgical blood loss thereby increasing the incidences of blood transfusions. Hypothermia has been categorized as either: mild (32.2° to 35° C.); moderate (27° and 32.2° C.); or severe (<80° F. or 27° C.) based on core body temperature as opposed to tactile or skin surface temperature readings that often belie the onset of hypothermia. Hypothermia based upon core body temperature assay often manifest in the symptoms of clinical depression, shivering, and increased pulse and metabolic rates, dysarthria, ataxia, and apathy. If the core body temperature goes below 90° F. or 32.2° C. the body loses its ability to shiver and re-warm spontaneously.

Whole body cooling is used clinically to protect the brain in some cardiac and neurologic patients. Hyperthermia (>2.0° C.) is also associated with adverse medical effects. Body core temperatures that are outside homeostatic limits are maybe afflicted with significant illness, which contributes or may cause their temperature deviation but temperature change itself may contribute to system dysfunction, however, many occupations expose workers to extremes in temperature by environmental exposure and not otherwise associated with an illness.

Core body temperature is normally regulated by the brain primarily by the hypothalamus. The brain integrates thermal inputs from: the skin surface; the lower neural centers; and the deep tissues with threshold temperatures that trigger each thermoregulatory response. This temperature information is generally processed in the spinal cord and brain stem prior to its receipt by the hypothalamus, however, there are some thermoregulatory responses that originate solely from the spinal cord.

It is well known in the art that the treatment of core temperature changes, either hypothermia or hyperthermia can be either passive or active. One common treatment of hypothermia is submersion in warm water (104° F. or 40° C.) however this is often dangerous because core temperatures may continue to fall as the surface is heated. More practical methods that are used in the art include heated inspiratory air (oxygen), warmed IV solutions, application of warmed blankets, and for more severe cases body cavity lavage with warm saline, and warming with extracorporeal blood circulation with or without cardiac bypass. Hyperthermia is treated with medications and surface cooling with alcohol baths or cooling blankets, however, it appears that the cooling of inspiratory air for treatment of hyperthermia is not known.

The human lungs perform vital functions in the human body. The most important lung function is of course to take in oxygen and remove carbon dioxide to and from the pulmonary blood flow through the lungs. The air taken in with each breath enters the lungs by the main windpipe called the trachea. The trachea then branches into two main tubes called the bronchi supplying the right and left lung, respectively. These bronchi branch 22 additional times to form more than 100,000 smaller tubes called bronchioles and collectively containing more than 300 million air sacs called alveoli which average 0.3 mm in diameter.

The surface area of the lungs of the average sized person (70 kg or 154 lbs) has been calculated to equal 900 ft², which is larger than the surface area of a tennis court. In contrast the average sized person's skin surface area is only 1.8 m² (19.4 ft²). Because the walls of these air sacs are 1/50th the thickness of tissue paper and are bathed in blood by millions of capillaries, there is an easy and efficient exchange of temperature between the body and the environment through the inhalation of air.

Simple calorimetry and the physics of thermal conductivity make it readily apparent that the potential heat exchange with the human body through the pulmonary system as opposed to the skin is 46 times greater based upon surface area contact alone. Furthermore, the physiology of the human body is such that often times the brain and spinal cord thermo-regulatory response to the skin causes a reduced circulation of blood to the extremities including the skin, thus further reducing the possibility of controlling core temperatures by skin exposure to heating and cooling devices. The pulmonary system cannot and does not react in the same manner since oxygenation of the blood by the lungs must continue or death would result. Therefore by heating inspiratory air in cases of hypothermia and cooling inspiratory air in cases of hyperthermia will directly affect body core temperatures. It should be noted that the thermal efficiency of the lungs is so high that regardless of the temperature of inspired air, the exhausted air from the lungs (except in extreme cases where body core temperatures are near fatal) is constant which is due in large measure to the evaporative and convective heat loss ability of the lungs. Pulmonary evaporative heat loss amounts to an average of one-third or more of the average person's entire evaporative heat loss depending upon the lung capacity of the individual, the atmospheric pressure, relative humidity, clothing and the respiration rate.

This body temperature heat exchange is calculated using the formula: S=(M+W)+R+C+(Cresp+Eresp)+E. A positive value for any of these terms signifies that the body core has gained heat, and a negative value indicates heat loss from the body core. Each of the terms represents an energy transfer rate. These rates are often normalized values to body surface area.

In the formula term S is the Heat Storage Rate. If the S value is zero then the body is in considered to be in thermal equilibrium with heat gain being balanced by heat loss. If the S value is positive then the body is gaining heat at the rate indicated by the value of S.

In the formula term M is Metabolic Rate. The rate of metabolism depends directly on the rate and type of external work currently being demanded. The W term is External Work Rate which is the amount of energy that is converted from biochemical energy to mechanical work. The W value is typically only about 10% of M. The W value is generally small relative to the other heat exchanges found in industrial applications so therefore the value is generally ignored.

The R term is the Radiant Heat Exchange Rate (Radiation). This is the rate of heat transfer by radiation which depends on the average temperature of the surrounding solid surfaces, skin temperature, and clothing.

The C term is the Convective Heat Exchange Rate (Convection). This is the exchange of heat between the skin and the surrounding air. The direction of heat flow depends on the temperature difference between the skin and air. If the ambient air temperature is greater than the skin's then the C term is positive and heat flows from the air to the skin. The rate of convective heat exchange depends on the magnitude of the temperature difference, the amount of air motion, and clothing.

The Cresp term is the Rate of Convective Heat Exchange by Respiration. The fact that air is moved in and out of the lungs, which have a surface area on average 46 times that of the skin, there is a huge opportunity to gain or lose heat by lung convection. The rate of heat exchange depends on the air temperature and volume of air inhaled. Adding to the lungs massive potential for heat exchange is the Eresp term which is the Rate of Evaporative Heat Loss by Respiration. Again the incredibly large surface area of the lungs provides an additional opportunity to lose heat with the pulmonary system (lungs) by evaporation. The rate of Eresp heat exchange depends on the air humidity and volume of air inhaled.

The E term is the Rate of Evaporative Heat Loss which for an average individual amounts to about ⅔ of the total evaporative heat loss of the body. Sweat on the skin surface will absorb heat from the skin when evaporating into the air. The process of evaporation cools the skin and, in turn, the body. The rate of evaporative heat loss depends on the amount of sweating, air movement, ambient humidity, and clothing, all of which can vary widely from one individual to the next.

The goal in design and usage of personal cooling and heating devices is to maintain the storage rate (S) at zero by controlling body surface temperatures or pulmonary air intake temperatures sufficient to remove (or add as the need may be) the heat generated by metabolism plus any heat gained from (or lost to) the environment through R+C. Again, because the surface area of the lungs are 46 times that of the skin, through convective and evaporative pulmonary heat transfer the cooling and/or heating of inspiratory air is not only the most effective, from an energy standpoint it is the most efficient.

There is a long felt need for a personal cooling and heating system that does not require the user to add or remove clothing. Military and law enforcement personnel have a need to cool themselves when is extreme heat conditions such as desert environment. More often than not, these individuals are required to wear a heavy uniform and ballistic protection gear. In yet other situations, military and civilian personnel that are required to use Chem-Bio gear have no way to cool themselves and consequently they experience an effective use time that is generally no more than 40 minutes before they run the risk of passing out from heat exhaustion.

Other technologies utilize a cooling garment worn next to the skin generally over a thin undergarment (i.e. “silks” or thin T-shirt), that the user then is required to wear underneath all their other clothing and gear. These garments generally have plastic tubing with chilled liquid running through them. This is considered the state of the art technology, however these types of systems are very inefficient and have a very short (2-3 hour maximum) power capacity with only 200 watts of cooling capability on average.

These systems manifest the following limitations:
1. They require the chilling of a liquid (i.e. water or alcohol, etc.) and then the pumping of the chilled liquid through the garment which requires additional power consumption in addition to the cooling process;
2. The user has to wear an additional garment, which in turn creates an additional insulating factor that tends to hold the user's own body heat next to or within their body and ultimately results in an additional thermal burden;
3. The garment is bulky and adds to the bulk of the user reducing their already limited movement capability;
4. The liquid thermal transfer medium and garment together add significant additional weight to the user, which in turn increases the heat generated by the user in doing the additional work of carrying this added weight, which in turn reduces the effectiveness of these types of systems;
5. The heat transfer ratio between the chilled liquid in the tubing and through the inner fabric mesh of the garment then through the recommended T-shirt and finally onto the surface of the skin is extremely inefficient.
6. The coefficient of heat transfer between human skin and a cooled surface results in physiological changes that reduce the efficiency of heat transfer. When human skin is exposed to cool or cold temperatures the body in an act of preservation of core temperature automatically allows the outer skin layers to remain cooler than the inner. This anomaly thereby reduces the heat transfer between the outer skin and the liquid tubing. In other words, because the human body by its nature reacts to temperature changes in the environment a cooling system of this type would need to sense this physiological change and adjust the cooling liquid temperature to compensate. This in turn would require even more power and thus reduce the systems overall operating time. There does not appear to be any prior art cooling systems that are capable of doing this.
7. Only about ⅓ of the surface area of the liquid filled tubing actually transfers energy to the body while the other ⅔ is in essence cooling the outer clothing and gear and ultimately the ambient air.
8. These systems also have hoses and connectors that must go through outer clothing, ballistic protection and ultimately compromise the protection of Chem-Bio suits to provide the chilled liquid to the garment.
9. Insofar as the garment must be worn underneath all other clothing, the user cannot easily remove the extra weight and thermal burden at will, such as at times when the battery power runs out and the system is no longer providing any cooling function. Consequently the user is simply stuck with this extra burden, which often is a serious if not fatal threat to the user's life.

There are no acceptable prior art heat stress and cold weather exposure relief systems for individuals, such as soldiers, operating in hot and cold environments for extended periods of time. Desert conditions for example often place individuals in a heat stress environment during the daylight hours and in severe cold during the nighttime. Heat stress can result in sweating, fatigue, dehydration, dizziness, hot skin temperature, muscle weakness, increased heart rate, heat rash, fainting, injuries, weight loss, heat stroke, heat exhaustion, and even death. The risk of heat stress is even greater for those wearing nuclear, biological and chemical (NBC) protective clothing, as well as aircrew personnel wearing flight gear. Cold weather exposure can cause discomfort; pain; numbness; cardiac, circulatory and respiratory problems; diminished muscle function and performance; frostbite, and hypothermia which can lead to unconsciousness and death.

While a portable, lightweight, low power, personal cooling and heating system can reduce heat stress, reduce the adverse effects of cold exposure, improve performance, and reduce water consumption, current active and passive cooling systems fall short of meeting the minimum requirements for an optimal system.

Active personal cooling devices are well know in the prior art. Also active personal heating systems are known in the prior art. The prior art, however, seems to be devoid of a combination cooling and heating system functioning with any significant efficiency over longer periods of time. The current active cooling and heating systems, however, are too heavy, bulky, inefficient, and are effective for only a limited amount of time. These devices also consume too much power and use potentially dangerous materials such as lithium sulfur dioxide batteries or R134 a refrigerant. Passive cooling and heating systems use packets containing phase change chemicals, water or gel that require refrigeration, freezing or heating before use are not suitable to meet the needs of a user where refrigeration, freezing or heating of the passive cooling or heating components are unavailable such as in military field operations in hot, cold or combined hot and cold climatic conditions. The prior art active cooling and heating systems that have been developed, include:
1. U.S. Army PICS (Personal Ice-Cooling System) Problem: This system uses packed ice. The ice must be changed every 30 minutes, and users such as pilots and field deployed soldiers may not have access to ice to replenish the system.
2. U.S. Army PVCS (Portable Vapor Compression Cooling System) Problems: The total system is much too heavy (27 pounds); uses potentially dangerous lithium sulphur dioxide batteries, can't use vapor compression on non-level surfaces such as ships; R134a containers can rupture in high temperatures, exposure to liquid or vapor refrigerant can cause frostbite, high exposure to fumes can cause central nervous system depression, irregular heartbeat and suffocation.
3. U.S. Army ALMCs (Advanced Lightweight Microclimate Cooling System) Problems: A voltage delay phenomenon can cause lithium sulphur dioxide batteries not to start especially after storage; the batteries can vent toxic sulphur dioxide gas that can cause respiratory distress and burns if there is accidental electrical charging, puncturing or application of heat. The batteries are not rechargeable, cannot be exposed to high temperatures, are very reactive with water and cannot be opened, punctured or crushed.
4. IMCC (Integrated Mesoscopic Cooling Circuits) (DARPA) Problem: Insufficient cooling.
5. Absorption/Evaporative Cooling (DARPA). Problem: According to Roger Masadi at the Natick Soldier Center, typical desiccants only adsorb about 20 percent of their weight in water, and the cooling density is approximately the same as ice.
6. NASA and U. S. Air Force (APECS) Aircrew Personal Environmental Control System Problem: This system is too bulky for infantry soldiers.
7. Life Enhancement Technologies Problem: The ice water mixture for the cooling unit must be replenished.

While each of these prior art personal cooling and heating systems which fulfill their respective particular objectives and requirements, and are most likely quite functional for their intended purposes, it will be noticed that none of the prior art cited disclose an apparatus and/or method that is portable, rugged, and lightweight and that can be used in any orientation or used as a belt-mounted system, gas mask, bio-hazard suit or a backpack, to meet the operational requirements of the user. Also, the prior art cannot provide several continuous hours of operation without the burden of carrying the extra weight of cooling liquids and the additional garments that in actuality only exacerbate the thermal demands of a user.

As such, there apparently still exists the need for new and improved personal cooling and heating system to maximize the benefits to the user and minimize the risks of injury from its use. The current invention addresses all of these issues to provide a technology that provides a much more effective, efficient and user friendly system that does not require additional thermal burden of additional garments and carrying the weight of cooling liquids.

The current invention takes advantage of the thermally high efficient surface area of the human lungs to control the body's core temperature by providing chilled or heated air directly to the user's lungs by the user's own breath. The pulmonary system then further regulates the body's core temperature by cooling or heating, as the case may be, the blood carried through the body after passing through the lungs and receiving the heated or cooled air. The coefficient of thermal conductivity from a cooling or heating source to air is among the highest known and the heat exchange surface area of the human lung is on average 900 ft², thereby minimizing, if not eliminating, thermal waste. The lungs surface itself is wet which results in a 100% absorption of any heat or cooled air temperature differential in the air contained in a user's breath by convection and evaporation.

In this respect, the present invention disclosed herein substantially corrects these problems and fulfills the need for such a device.

### DISCLOSURE OF TIE INVENTION

In view of the foregoing limitations inherent in the known types of personal cooling and heating systems now present in the prior art, the present invention provides an apparatus that has been designed to provide the following features for a user:
A virtually unlimited amount of thermal power of adjustable heating or cooling per hour.
Direct thermal impact on the body core temperature by cooling or heating inspiratory air.
Automatically adjusting the temperature of the inspiratory air in direct response to the body core temperature
Extremely lightweight adding little to no further thermal burden to the user.
Minimum of two hours of continuous operation in battery operated devices and unlimited time in external power sources.
On-demand cooling and heating.
2000 failure-free hours.
Self or externally powered.
Resistant to chemical agents.
Easily decontaminated.
Easy to maintain with a minimum of hand tools.
Safe to the touch.
Power supply compatibility with other flight line or aircraft systems.
Compliance with electromagnetic compatibility and interface (EMC/EMI) requirements.
The system can be operated and recharged by ground power cart or aircraft power.

These features are improvements which are patently distinct over similar devices and methods which may already be patented or commercially available. As such, the general purpose of the present invention, which will be described subsequently in greater detail, is to provide a field designed apparatus and method of manufacture that incorporates the present invention. There are many additional novel features directed to solving problems not addressed in the prior art.

To attain this the present invention generally comprises six major components: 1) A user interface whereby when a user draws a breath while this invention is in use the inspiratory air comes principally from the device; 2) A housing connected to the user interface; 3) A cooling means contained within the housing such that when a user draws a breath the drawn air is thereby drawn into the housing where it is cooled prior to its exiting the housing through the user interface thereafter entering the user's body and ultimately being drawn into the lungs; 4) A heating means contained within the housing such that when a user draws a breath the drawn air is thereby drawn into the housing where it is heated prior to its exiting the housing through the user interface thereafter entering the user's body and ultimately being drawn into the lungs; 5) A control means to regulate the cooling means and the heating means; and 6) A valve means to prevent expired air from entering the housing.

These together with other objects of the invention, along with the various features of novelty which characterize the invention, will be pointed out with particularity in the claims which will be annexed to and forming a part of the full patent application once filed. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

FIG. 1 is perspective view of the battery powered Portable Pulmonary Body Core Cooling and Heating System attached to a gas mask for operation.

FIG. 1A is perspective view of the battery powered Portable Pulmonary Body Core Cooling and Heating System gas mask embodiment partially disassembled.

FIG. 2 is an exploded perspective view of the battery powered Portable Pulmonary Body Core Cooling and Heating System.

FIG. 2A is an exploded perspective view of the battery powered Portable Pulmonary Body Core Cooling and Heating System of FIG. 2 as viewed from the opposite direction.

FIG. 3 is a perspective view of the battery powered Portable Pulmonary Body Core Cooling and Heating System.

FIG. 4 is an exploded perspective view of the externally powered Portable Pulmonary Body Core Cooling and Heating System.

FIG. 5 is a perspective view of the external heating or cooling source Portable Pulmonary Body Core Cooling and Heating System.

### BEST MODES FOR CARRYING OUT THE INVENTION

### I. Preferred Embodiments

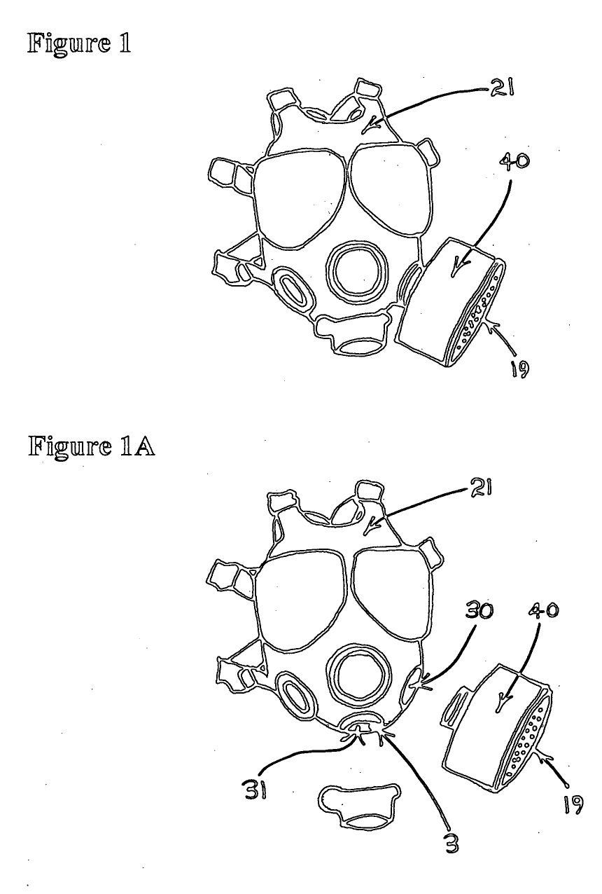

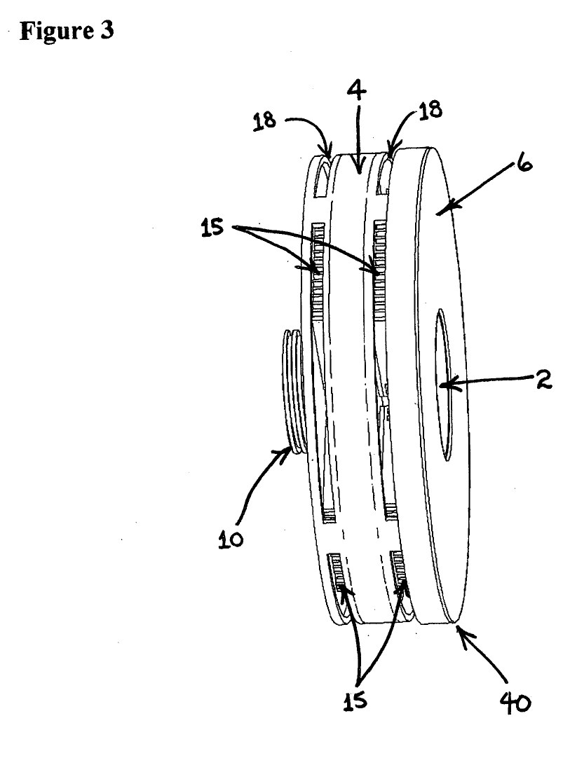

With reference now to the drawings, and in particular to FIGS. 1-5 thereof, a new and novel Portable Pulmonary Body Core Cooling and Heating System (40) embodying the principles and concepts of the present invention is depicted in these drawings as comprising six major components, the User Interface (10), the Housing (4), the Cooling Means and the Heating Means which together generally comprise the Temperature Exchange Module (11), the Control Means which is comprised of the Micro Controller Display and Keypad (12) and the Micro Controller (16) and the Valve Means which is comprised of an Intake Valve (30) and an Exhaust Valve (31).

### GENERAL DESCRIPTION OF REFERENCE NUMERALS IN THE DESCRIPTION AND DRAWINGS

Any actual dimensions listed are those of the preferred embodiment. Actual dimensions or exact hardware details and means may vary in a final product or most preferred embodiment and should be considered means for so as not to narrow the claims of the patent.

List and Description of component parts of the invention:
(1) Reversible Thermoelectric Cooler (TEC) Modules.
(2) Intake Air Orifice
(3) Temperature Sensor
(4) Housing
(5) Silicon Sealing Gasket
(6) Filter End Plate
(7) Battery
(8) Electric Heating Strip
(9) Silicon Sealing Gasket Seat
(10) User Interface
(11) Temperature Exchange Module
(12) Micro Controller Display and Keypad
(13) Cooling Fans
(14) Micro Controller and Battery Housing
(15) Cooling Fins
(16) Micro Controller
(18) Air Exhaust Port
(19) Filter
(21) Gas Mask
(22) External Power Source
(23) External Heat Source
(24) External Cooling Source
(30) Intake Valve
(31) Exhaust Valve
(40) Portable Pulmonary Body Core Cooling and Heating System

### I. Detailed Description of the Most Preferred Embodiment

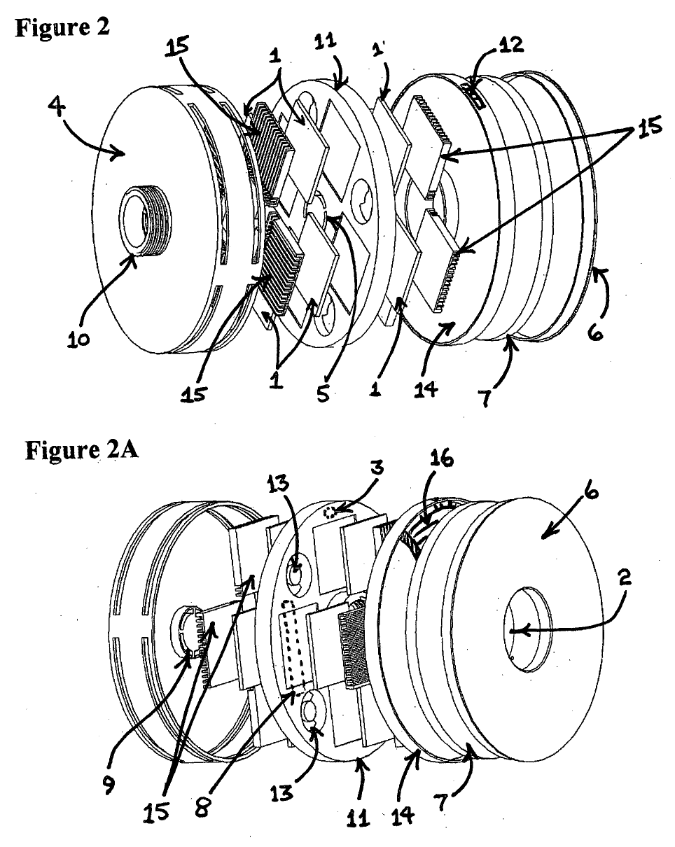

The user screws into a standard sized gas mask the Portable Pulmonary Body Core Cooling and Heating System (40) as depicted in FIGS. 1 and 1A.
Cooling or heating is started by activating the power switch of the Micro Controller Display and Keypad (12) on the Portable Pulmonary Body Core Cooling and Heating System (40) as depicted in FIGS. 2 and 2A. The user can adjust the cooling or heating rate by a wireless or wired remote control.
For cooling, the Micro Controller (16) checks the capacity of the Battery (7) and begins to monitor the system's Temperature Sensors (3). While monitoring the Temperature Sensors (3), the Micro Controller(16) automatically makes adjustments to the speed of the Cooling Fans (13) and the temperature of the Reversible Thermoelectric Cooler (TEC) Modules (1) to meet the user's cooling and/or heating requirements with the most power-efficient settings.
The Micro Controller (16) powers up the Reversible Thermoelectric Cooler (TEC) Modules (1) and continually monitors the power supply drain and capacity. The Reversible Thermoelectric Cooler (TEC) Modules (1) provide cooling or heating (per the user's selection) by changing the temperature of the Temperature Exchange Module (11) thus taking ambient air changing the temperature of that air as it is drawn by the user's inhalation internally through channels inside the Temperature Exchange Module (11) through the Portable Pulmonary Body Core Cooling and Heating System (40) to the user.
For heating in more extreme situations where the Reversible Thermoelectric Cooler (TEC) Modules (1) are unable to meet the demand a flexible Electric Heating Strip (8) is thereby activated causing the Temperature Exchange Module (11) to generate additional heat.
The Battery (7) can be exchanged or recharged after two or more hours of operation depending upon user settings and concomitant energy demands.
As a user breaths using the Portable Pulmonary Body Core Cooling and Heating System (40) air is drawn into the device through the Filter (19) by means of the Intake Air Orifice (2) of the Filter End Plate (6) where the air then enters into the center of the Temperature Exchange Module (11) where the air temperature is changed by direct heat transfer to either a cooler or warmer temperature depending upon the needs of the user and travels within channels inside the Temperature Exchange Module (11) and then exits the Portable Pulmonary Body Core Cooling and Heating System (40) through a one way Intake Valve (30) into the lungs of the user as the user inhales, then the exhaled air exits the Gas Mask (21) through a one way Exhaust Valve (31) into the ambient air bypassing the Portable Pulmonary Body Core Cooling and Heating System (40) upon exhaust.

### Description of Components of The Pulmonary Body Core Cooling and Heating System of the Most Preferred Embodiment

The Pulmonary Body Core Cooling and Heating System has four main components:

### 1) Cooling Unit (CU):

In the Preferred Embodiment as depicted in FIGS. 1,2 and 3 the Cooling Unit (CU) is comprised of eight Reversible Thermoelectric Cooler (TEC) Modules (1) attached to the Temperature Exchange Module (11) such that the cold side of the eight Reversible Thermoelectric Cooler (TEC) Modules (1) thereby chill the Temperature Exchange Module (11) with the hot side of the eight Reversible Thermoelectric Cooler (TEC) Modules (1) each has a corresponding Cooling Fin (15) attached thereto to act as a heat sink to draw the heat away from the Reversible Thermoelectric Cooler (TEC) Modules (1); the eight Cooling Fins (15) are further aided in disbursing heat by the four Cooling Fans (13) which is activated by the Micro Controller (16) that is electrically and/or electronically connected to internal Temperature Sensors (3) in the Temperature Exchange Module (11) and the Air Exhaust Port (18).

### 2) Heating Unit (HU):

In the Preferred Embodiment as depicted in FIGS. 2 and 2A the Heating Unit uses the following components of the Cooling Unit: the Electric Heating Strip (8); the eight Reversible Thermoelectric Cooler (TEC) Modules (1); and the Micro Controller (16) electrically and/or electronically connected to: the Temperature Sensors (3). The Electric Heating Strip (8) will evenly distribute heat over the Temperature Exchange Module (11) to provide the optimal heat transfer to the user.

### 3) Power Supply (PS):

In the Preferred Embodiment as depicted in FIGS. 1,2 and 3 the Battery (7) for both the Cooling and Heating Units are generally off-the-shelf, rechargeable Lithium Ion batteries.

### 4) Gas Mask (21):

In the Preferred Embodiment as depicted in FIGS. 1,2 and 3 the system will be used with a Gas Mask (21) to provide a relatively air tight connection between the Portable Pulmonary Body Core Cooling and Heating System (40) and the user. The Air Exhaust Port (18) contains at least one Temperature Sensor (3) unit that monitors the temperature of the exhausted air that leaves a user's lungs and body after taking a breath and send this information to the Micro Controller (16) where it automatically processes and calculates the user's body core temperature and automatically adjusts the cooling and/or heating of the device to meet the demands of the user to maintain the user's core body temperature within normal range.

### Other Embodiments

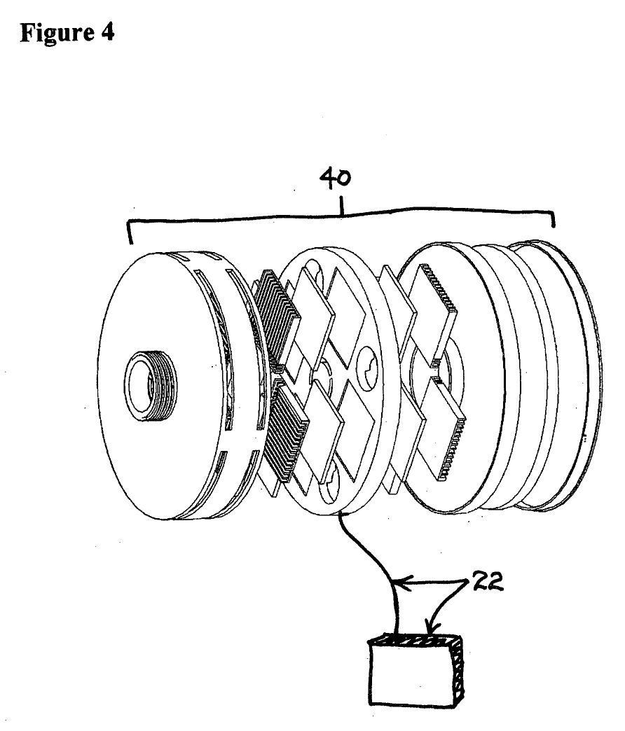

As depicted in FIG. 4 the Portable Pulmonary Body Core Cooling and Heating System (40) utilizes an External Power Source (22) thereby eliminating the need for a mounted battery on the unit. The power source can be AC or DC, portable or fixed depending upon the user's needs.

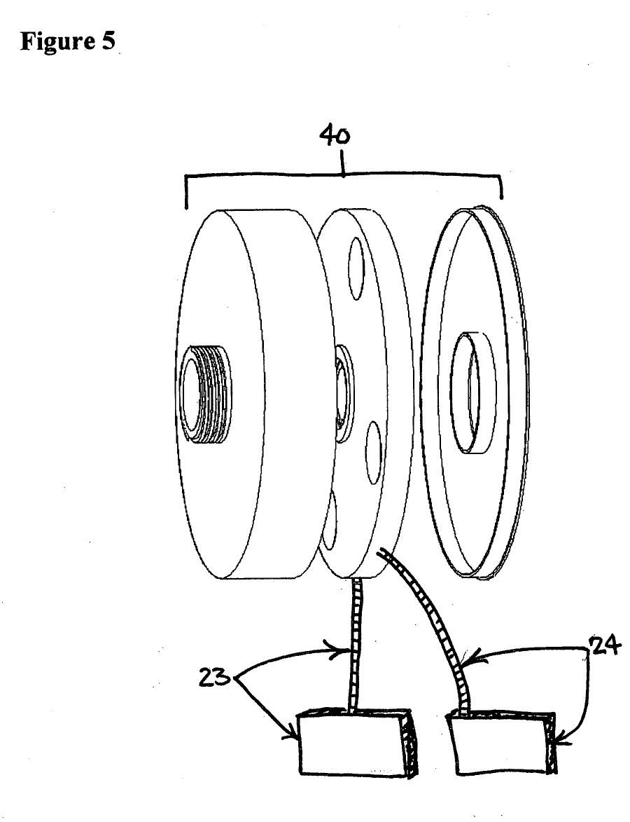

As depicted in FIG. 5 the Portable Pulmonary Body Core Cooling and Heating System (40) utilizes an External Heating Source (23) and an External Cooling Source (24) thereby eliminating the need to have the weight of the power source and cooling and heating means in the unit. It would be obvious to one skilled in the art to have a device that just cooled or a device that just heated the air being breathed by a user.

It would be obvious to one skilled in the art to use fuels such as odorless, clean-burning, non-smoking liquid fuels such as liquid benzine, pure white gasoline or lighter fluid as a replacement for the Electric Heating Strip (8) and/or the Reversible Thermoelectric Cooler (TEC) Modules (1). The burner would be installed externally. The drawbacks of using the burner are that the user would be required to carry a flammable liquid, would have to light the burner to ignite it. It would be possible to design a burner with an electronic ignition and controls that would not require the user to manually light it or shut it off. This type of design would provide the most heat for the weight of the system but would potentially be very dangerous for use in such activities as flight line maintenance since they are typically working in proximity to aircraft fuel vapors.

While my above descriptions of the invention, its parts, and operations contains many specificities, these should not be construed as limitations on the scope of the invention, but rather as exemplifications of present embodiments thereof. Many other variations are possible, for example, other embodiments, shapes, and sizes of the device can be constructed and designed to work by the principles of the present invention; various materials, colors and configurations can be employed in the device's design that would provide interesting embodiment differences to users. The power supply to the unit may also be photovoltaic, as well as many other obvious variations. There are many different means of cooling from the use of ice, liquid nitrogen, dry ice, external cool or cold ambient air to standard compressed refrigeration techniques and many others that would all be obvious cooling means.

It would also be obvious to use some other means of connecting the device to the user such as a mouthpiece or a helmet such as in a bio-hazard outfit.

Accordingly, the scope of the invention should be determined not by the embodiments illustrated, but by the claims and their legal equivalents as filed herewith.

Having described my invention, I claim:

1. A portable pulmonary body core cooling and heating system comprised of
a user interface means wherein when a user breaths, the air that is breathed in by the user passes through the user interface means into the user's nose and/or mouth during breathing;
a cooling means attached to the user interface means wherein the air breathed in by the user passes first through the cooling means cooling the air thereby and then passes through the user interface means into the user's nose and/or mouth during breathing;
an intake valve means attached to the user interface means and the cooling means wherein when a user breathes in air the intake valve means allows cooled air from the cooling means to enter into the user interface means prior to entering into the user's nose and/or mouth during an inhalation phase of breathing and upon an exhaustion phase of a user's breathing the intake valve means does not permit the exhausted air to enter or pass through the cooling means;
an exhaust valve means attached to the user interface means wherein when a user exhausts air during breathing the exhaust valve means allows the exhausted air to exit the user interface means during the exhaustion phase of the user's breathing and once the exhaustion phase has ended the exhaust valve means does not permit any exhausted air to reenter the user interface means in order that the air that will enter the user's nose and/or mouth in the next inhalation phase of the user's breathing will have been cooled by passing through the cooling means prior to the next inhalation phase of the user's breathing;
a temperature control means attached to the cooling means wherein the temperature control means regulates the extent to which the air is cooled by the cooling means; and
a power supply means electrically attached to the cooling means and the temperature control means.

2. A portable pulmonary body core cooling and heating system comprised of
a user interface means wherein when a user breaths, the air that is breathed in by the user passes through the user interface means into the user's nose and/or mouth during breathing;
a heating means attached to the user interface means wherein the air breathed in by the user passes first through the heating means heating the air thereby and then passes through the user interface means into the user's nose and/or mouth during breathing;
an intake valve means attached to the user interface means and the heating means wherein when a user breathes in air the intake valve means allows heated air from the heating means to enter into the user interface means prior to entering into the user's nose and/or mouth during an inhalation phase of breathing and upon an exhaustion phase of a user's breathing the intake valve means does not permit the exhausted air to enter or pass through the heating means;
an exhaust valve means attached to the user interface means wherein when a user exhausts air during breathing the exhaust valve means allows the exhausted air to exit the user interface means during the exhaustion phase of the user's breathing and once the exhaustion phase has ended the exhaust valve means does not permit any exhausted air to reenter the user interface means in order that the air that will enter the user's nose and/or mouth in the next inhalation phase of the user's breathing will have been heated by passing through the heating means prior to the next inhalation phase of the user's breathing;
a temperature control means attached to the heating means wherein the temperature control means regulates the extent to which the air is heated by the heating means; and
a power supply means electrically attached to the heating means and the temperature control means.

3. A portable pulmonary body core cooling and heating system comprised of:
a user interface means wherein when a user breaths, the air that is breathed in by the user passes through the user interface means into the user's nose and/or mouth during breathing;
a combination cooling and heating means attached to the user interface means wherein the air breathed in by the user passes first through the combination cooling and heating means either cooling or heating the air thereby and then passes through the user interface means into the user's nose and/or mouth during breathing;
an intake valve means attached to the user interface means and the combination cooling and heating means wherein when a user breathes in air the intake valve means allows cooled or heated air from the combination cooling and heating means to enter into the user interface means prior to entering into the user's nose and/or mouth during an inhalation phase of breathing and upon an exhaustion phase of a user's breathing the intake valve means does not permit the exhausted air to enter or pass through the combination cooling and heating means;
an exhaust valve means attached to the user interface means wherein when a user exhausts air during breathing the exhaust valve means allows the exhausted air to exit the user interface means during the exhaustion phase of the user's breathing and once the exhaustion phase has ended the exhaust valve means does not permit any exhausted air to reenter the user interface means in order that the air that will enter the user's nose and/or mouth in the next inhalation phase of the user's breathing will have been cooled or heated by passing through the combination cooling and heating means prior to the next inhalation phase of the user's breathing;
a temperature control means attached to the combination cooling and heating means wherein the temperature control means regulates the extent to which the air is either cooled or heated by the combination cooling and heating means; and
a power supply means electrically attached to the combination cooling and heating means and the temperature control means.

4. The portable pulmonary body core cooling and heating system of claim 1 wherein the user interface means is a gas mask.

5. The portable pulmonary body core cooling and heating system of claim 1 wherein the cooling means is comprised of at least one reversible thermoelectric cooler module electrically attached to and activated by a reversible direct current of electricity that is pulsed from the temperature control means.

6. The portable pulmonary body core cooling and heating system of claim 1 wherein the cooling means is comprised of at least one of the cooling agents selected from the group consisting of frozen water, frozen carbon dioxide, liquid nitrogen, ambient air, reversible thermoelectric cooler module and compressed refrigerants.

7. The portable pulmonary body core cooling and heating system of claim 1 wherein the temperature control means is comprised of a vital signs detection means capable of processing a user's vital signs to determine if a user's body core temperature is rising thereby automatically adjusting the cooling means to cool the air being breathed in by the user.

8. The portable pulmonary body core cooling and heating system of claim 1 wherein the temperature control means is comprised of a user controlled manual temperature adjustment means.

9. The portable pulmonary body core cooling and heating system of claim 2 wherein the user interface means is a gas mask.

10. The portable pulmonary body core cooling and heating system of claim 2 wherein the heating means is comprised of at least one reversible thermoelectric cooler module electrically attached to and activated by a reversible direct current of electricity that is pulsed from the temperature control means.

11. The portable pulmonary body core cooling and heating system of claim 2 wherein the heating means is comprised of at least one of the heating agents selected from the group consisting of liquid benzine, pure white gasoline, lighter fluid, reversible thermoelectric cooler module and electric heating strip.

12. The portable pulmonary body core cooling and heating system of claim 2 wherein the temperature control means is comprised of a vital signs detection means capable of processing a user's vital signs to determine if a user's body core temperature is rising thereby automatically adjusting the cooling means to cool the air being breathed in by the user.

13. The portable pulmonary body core cooling and heating system of claim 2 wherein the temperature control means is comprised of a user controlled manual temperature adjustment means.

14. The portable pulmonary body core cooling and heating system of claim 3 wherein the user interface means is a gas mask.

15. The portable pulmonary body core cooling and heating system of claim 3 wherein the combination cooling and heating means is comprised of at least one reversible thermoelectric cooler module electrically attached to and activated by a reversible direct current of electricity that is pulsed from the temperature control means.

16. The portable pulmonary body core cooling and heating system of claim 3 wherein the combination cooling and heating means is comprised of at least one of the heating agents selected from the group consisting of liquid benzine, pure white gasoline, lighter fluid, reversible thermoelectric cooler module and electric heating strip.

17. The portable pulmonary body core cooling and heating system of claim 3 wherein the combination cooling and heating means is comprised of at least one of the cooling agents selected from the group consisting of frozen water, frozen carbon dioxide, liquid nitrogen, ambient air, reversible thermoelectric cooler module and compressed refrigerants.

18. The portable pulmonary body core cooling and heating system of claim 3 wherein the temperature control means is comprised of a vital signs detection means capable of processing a user's vital signs to determine if a user's body core temperature is rising thereby automatically adjusting the combination cooling and heating means to either cool or heat the air being breathed in by the user.

19. The portable pulmonary body core cooling and heating system of claim 3 wherein the temperature control means is comprised of a user controlled manual temperature adjustment means.

20. The portable pulmonary body core cooling and heating system of claim 3 wherein the combination cooling and heating means is comprised of at least one of the heating agents selected from the group consisting of liquid benzine, pure white gasoline, lighter fluid, reversible thermoelectric cooler module and electric heating strip and the combination cooling and heating means is further comprised of at least one of the cooling agents selected from the group consisting of frozen water, frozen carbon dioxide, liquid nitrogen, ambient air, reversible thermoelectric cooler module and compressed refrigerants.
